(19) United States
(12) Patent Application Publication
(10) Pub. No.: US 2003/0143645 A1
(43) Pub. Date: Jul. 31, 2003

(54) Quinone reductase 2 and aldehyde dehydrogenase as therapeutic targets

(76) Inventor: Timothy A. Haystead, Chapel Hill, NC (US)

Correspondence Address: NIXON & VANDERHYE P.C., 8th Floor, 1100 North Glebe Road, Arlington, VA, 22201, US

(21) Appl. No.: 10/243,947

(22) Filed: Sep. 16, 2002

Related U.S. Application Data
(60) Provisional application No. 60/318,819, filed on Sep. 14, 2001.

(51) Int. Cl. G01N 33/53; G01N 33/569; C12Q 1/26; A61K 31/47

(52) U.S. Cl. 435/7.22; 514/313; 514/305; 435/25

(57) ABSTRACT

The present invention relates, in general, to quinone reductase 2 (QR2) and aldehyde dehydrogenase (ALDH), and, in particular, to methods of screening compounds for their ability to modulate the activity of QR2 and/or ALDH and thereby to function as anti-malarial, anti-arthritic and/or anti-lupus agents. The invention further relates to the use of compounds that inhibit ALDH in the production of stem cells en masse.

This application claims priority from U.S. Provisional Application No. 60/318,819, filed Sep. 14, 2001, the entire content of which is incorporated herein by reference.

### TECHNICAL FIELD

The present invention relates, in general, to quinone reductase 2 (QR2) and aldehyde dehydrogenase (ALDH), and, in particular, to methods of screening compounds for their ability to modulate the activity of QR2 and/or ALDH and thereby to function as anti-malarial, anti-arthritic and/or anti-lupus agents. The invention further relates to the use of compounds that inhibit ALDH in the production of stem cells en masse.

### BACKGROUND

One third of the global human population is exposed to malaria with approximately 90% of cases occurring in sub-Saharan Africa and the remaining 10% occurring in south and southeast Asia and central and south America (World Health Organization Press, pages 49-53 (1999)). Malaria is caused by protozoan parasites of the genus Plasmodium (Kemp et al, Annu. Rev. Microbiol. 41:181-208 (1987), Weatherall et al, The anaemia of Plasmodium falciparum malaria, London (1993), Miller, Science 257:36-37 (1992)). The life cycle begins when an infected female mosquito bites her prey injecting it with parasite-containing (sporozoite) saliva. The sporozoites enter liver cells and multiply to form a different stage called merozoites. After 5 days, ˜40,000 merozoites are released into the blood stream where they enter red blood cells (RBC). The RBC provide the parasite with a safe haven from the host's immune system. The parasites grow by ingesting host hemoglobin (Hb) and divide to produce about 8-16 daughter merozoites. The merozoites burst from the RBC, releasing cell debris, causing a febrile episode in the host. Within minutes the merozoites invade new RBC and the cycle continues. After several cycles some of the intra-erythrocytic parasites develop into sexual stages, the gametocytes. The gametes are ingested when a mosquito bites an infected individual. They mate in the gut of the insect, pass through the gut wall, develop into sporozoites and migrate to the salivary glands to be passed onto another individual. While the blood forms of the parasite cause most of the pathology of the disease, they are also the stages that are most susceptible to attack by anti-malarial drugs.

Most of the available anti-malarial drugs kill the parasite as it resides within the RBC. Quinoline-containing anti-malarial drugs (CQ's), such as chloroquine (CQ), quinine (Q) primaquine (PQ) and mefloquine (MQ) (see FIG. 4), are a vital part of the chemotherapeutic armory against malaria. Sadly, many species of Plasmodium have become resistant to these drugs. Therefore, there is an urgent need to understand both the molecular mechanisms of the CQ's as well as the mechanisms by which the malarial parasite has developed resistance. By understanding these processes, novel quinoline anti-malarials can be designed that circumvent the problem of resistance. Surprisingly, despite a successful 50 year history, the mechanism of action of the CQ and its derivatives remains elusive. In the case of CQ itself, one hypothesis suggests the drug acts by interfering with the digestion of hemoglobin (Hb) (Goldberg et al, Proc. Natl. Acad. Sci. 87:2931-2935 (1990), Vander Jagt et al, Molec. Biochem. Parasitol. 18:389-400 (1986), Gabay et al, Parasitol 108:371-381 (1994)). CQ is dibasic and diffuses down a pH gradient to accumulate (1000-fold) in the acidic vacuole of the parasite (Yayon et al, EMBO J. 3:2695-2700 (1984), Homewood et al, Nature 235:50-52 (1972)). In this food vacuole, Hb is degraded, releasing free heme as a toxic bi-product. The high intravacuolar [CQ] has been proposed to interfere with the detoxification of heme resulting in a build up of heme to lethal levels, killing the parasite with its own metabolic waste. However, the structurally related anti-malarials, MQ, PQ and Q, are not concentrated so extensively in the food vacuole casting doubt on the pH hypothesis (Wellems, Nature 355(6356):109-109 (1992), Geary et al, Biochem. Pharmacol. 35(21):3805-3812 (1986), San George et al, Biochim. Biophys. Acta. 803(3):174-181 (1984), Desneves et al, 82(2):181-194 (1996), Peters, Trop. Doct. 17(1):1-3 (1987), Somasundaram et al, Biochem. J. 309(Pt 3):725-729 (1995)). Foley and colleagues recently identified an alternative target for CQ, lactate dehydrogenase (PfLDH) in P. falciparum (Foley et al, J. Biol. Chem. 269(9):6955-6961 (1994), Benting et al, Mol. Biochem. Parasitol. 88(1-2):215-224 (1997), Dunn et al, Nat. Struct. Biol. 3(11):912-915 (1996)). However, although CQ was shown to bind to PfLDH in the cofactor binding site, it does not inhibit activity (Dunn, Nat. Struct. Biol. 3(11):912-915 (1996)). Clearly, therefore, other mechanisms of action must exist to explain the pharmacological effects of this important class of drugs.

In addition to their anti-malarial actions, the CQ's have therapeutic value in the treatment of lupus erythematosus and rheumatoid arthritis (for review see Rynes, British J. Rheumatology 36:799-805 (1997) and Colman, Annu. Rev. Biochem. 52:67-91 (1983) and references cited therein). The efficacy of CQ's in the treatment of these diseases was discovered serendipitously following the prophylactic treatment of some 3-4 million soldiers for malaria in World War II (Beek et al, Dermatolo. 19:1-11 (1971)). The CQ's have become the parenteral drugs of choice for treating the cutaneous manifestations of lupus as well as a variety of other dermatoses. In arthritis, in responsive patients, long term treatment with CQ's can bring about significant improvement of symptoms to complete remission. A major side effect and contraindication of CQ's in the treatment of both conditions, however, is the development of retinopathy which can lead to blindness if unchecked (Beek et al, Dermatolo. 19:1-11 (1971)), Rynes, British J. Rheumatology 36:799-805 (1997)). The cause of retinopathy is unknown as are the molecular mechanisms underlying the therapeutic actions of CQ in the treatment of lupus and arthritis.

The present invention results from the identification of two physiological targets for CQ's that explain both the action of these drugs as anti-malarial agents and their side effects.

### SUMMARY OF THE INVENTION

The present invention relates generally to quinone reductase 2 (QR2) and aldehyde dehydrogenase (ALDH). Specifically, the invention relates to methods of screening compounds for the ability to modulate the activity of QR2 and/or ALDH and thereby to function as anti-malarial agents. QR2 and/or ALDH modulators identifiable using the present screens can be used in the treatment of autoimmune diseases, including lupus and arthritis (e.g., rheumatoid arthritis), and other diseases/disorders amenable to treatment using CQ type drugs. The invention further relates to the use of compounds that inhibit ALDH, including CQ's, in the production of stem cells en masse.

Objects and advantages of the present invention will be clear from the description that follows.

### BRIEF DESCRIPTION OF THE DRAWINGS

FIGS. 1A-1C. Catching the mouse purine nucleotide binding proteome. Mouse extract was prepared and passed over 1 ml of gamma phosphate linked ATP-Sepharose containing 10-15 μmols/ml of linked ATP. Following washing, the column was eluted sequentially with the indicated nucleotides and fractions collected (10 ml). Column fractions were separated by 1D (FIG. 1A) or 2D (FIG. 1B) SDS-PAGE. In a separate experiment N6 linked ATP (10-15 μmol/ml) was used resulting in the recovery of relatively few proteins. The ATP eluate was also concentrated 100 fold and 10 μl analyzed by 2D SDS-PAGE. In mixed peptide sequencing experiments, an average of 6-12 Edman cycles were carried out. The mixed sequences were sorted and matched against the entire published protein or DNA data bases with the FASTF or TFASTF algorithms, respectively (Damer et al, J. Biol. Chem. 273:24396 (1998)). FIG. 1C shows protein identified in SWISS-PROT, NCBI or mouse EST databases, isoelectric point (pI), molecular weight (MW) and expectation score (e). Generally, in cases where mass spectrometry was employed, 3 to 4 peptides were sequenced to positively identify a protein in the annotated protein data bases using FASTS (Damer et al, J. Biol. Chem. 273:24396 (1998)). The experiment shown was repeated on several separate occasions with similar results.

FIGS. 2A-2C. Catching the human RBC and P. falciparum purine binding proteomes. Homogenates prepared from 1×10⁸ non-infected (FIG. 2A) or infected (FIG. 2B) RBC were passed in parallel over two channels containing 1 ml of ATP resin in the array. A selection of the bound proteins were characterized by either mixed peptide sequencing or mass spectrometry. All proteins were identified from multiple peptide sequence alignments using FASTS or FASTF. In the case of proteins identified in the infected cells, expectation scores (e) are given in FIG. 2C for P. falciparum and the next highest scoring human homolog.

FIGS. 3A and 3B. Testing the selectivity of chloroquines against the non-infected and infected purine binding proteome. In FIG. 3A, an ATP affinity array consisting of 4 parallel 1.0 ml microcolumns were charged equally with 1×10⁸ RBC. The array was washed extensively with high and low ionic strength buffers. Each channel was eluted with the indicated drugs. In FIG. 3B, a single ATP affinity array channel charged with 1×10⁸ infected red cells was eluted with increasing concentrations of CQ. Similar results were found following elution of infected cell charged ATP affinity arrays with PQ, 4AQ and MQ. Eluted proteins were characterized by SDS-PAGE and silver staining, then identified by peptide sequencing using mass spectrometry.

FIG. 4. The chemical structures of ATP and antimalarial compounds.

FIG. 5. Primaquine Sepharose selectively recovers hALDH1 and hQR2 from RBC and whole mouse extract. RBC or mouse extract charged PQ-Sepharose (1.0 ml) was eluted with the indicated drugs and purines. The eluted proteins were characterized by SDS-PAGE and silver staining, then identified by peptide sequencing using mass spectrometry.

FIGS. 6A-6C. The crystal structures of hALDH1 (FIG. 6A), PfLDH (FIG. 6B) and hQR2 (FIG. 6C). Coordinates for the structures were obtained from the protein structure data base and images created using RASMOL.

FIG. 7. Proteomics strategy for identification and validation of quinoline antimalarial drug targets.

In step 1, a cell or animal lysate is passed over columns of ATP-Sepharose in parallel, washed to remove non-specific proteins, and then proteins are eluted with quinoline antimalarials. In step 2, a cell or animal lysate is passed over quinoline antimalarial drug columns (HCQ, hydroxychloroquine-Sepharose; PQ, primaquine-Sepharose), washed to remove non-specific proteins and then proteins eluted with quinoline antimalarials. All proteins are then sequenced and identified by mass spectrometry. In step 3, the isolated proteins from steps 1 and 2 are assayed for biological activity in the presence of quinoline antimalarials.

FIGS. 8A-8C. Capture and analysis of the mouse purine nucleotide binding proteome. (FIG. 8A), Proteins were eluted from γ-linked ATP-Sepharose (charged with whole mouse extract) with the indicated nucleotides, resolved by 1-D SDS-PAGE, visualized by silver staining, and sequenced by mixed peptide sequencing (Damer et al, J. Biol. Chem. 273(38):24396-24405 (1998)) or mass spectrometry. (FIG. 8B), 2-D SDS-PAGE of the eluate from γ-ATP-Sepharose following elution with ATP (FIG. 8C), List of identified proteins that specifically bound γ-linked ATP-Sepharose. The proteins molecular weight (MW) and expectation score (e) are shown.

FIGS. 9A and 9B. Capture and analysis of the human RBC and P. falciparum purine nucleotide binding proteome. (FIG. 9A), Homogenates from non-infected RBCs or, (FIG. 9B), P. falciparum-infected RBCs were passed in parallel over columns containing γ-linked ATP-Sepharose, washed, and eluted with SDS. Proteins were resolved by SDS-PAGE, visualized by silver staining and identified by mixed peptide sequencing (Damer et al, J. Biol. Chem. 273(38):24396-24405 (1998)) or mass spectrometry. Expectation scores (e) are given for P. falciparum proteins and the next highest scoring human protein.

FIGS. 10A-10C. Identification of quinoline antimalarial binding proteins in the human RBC or P. falciparum purine nucleotide binding proteome. (FIG. 10A), Elution of γ-ATP-Sepharose charged with non-infected human RBC extract with the indicated drugs. (FIG. 10B), Elution of γ-ATP-Sepharose charged with P. falciparum-infected RBC extract with CQ or SDS. Proteins were resolved by SDS-PAGE, visualized by silver staining, and sequenced by mass spectrometry. (FIG. 10C), Identification of human ALDH1 and QR2 by FASTS. Peptide sequences shown were obtained by mass spectrometry and used to search the NCBI/Blast NR database using the FASTS algorithm (Mackey et al, Molecular and Cellular Proteomics 1.2:139-147 (2002)). The expectation score (e) for each protein is shown.

FIGS. 11A-11C. ALDH1 and QR2 are recovered and selectively eluted from PQ and HCQ-Sepharose. (FIG. 11A), PQ-Sepharose was charged with human RBC or mouse extract (indicated “mouse”) and eluted with the indicated drugs. The amount of NP-40 included in the binding and wash buffer is indicated. (FIG. 11B), HCQ-Sepharose was charged with human RBC extract and eluted with CQ. (FIG. 11C), Elution of RBC-extract charged PQ-Sepharose with NAD⁺, NMeH, and FAD⁺. The eluted proteins were resolved by SDS-PAGE, visualized by silver staining, and sequenced by mass spectrometry.

FIGS. 12A and 12B. QR2 and ALDH1 inhibitors have antimalarial activity in vitro. The growth of P. falciparum was measured in the presence of: (FIG. 12A) CQ (▪), MQ (▴), and PQ (♦). (FIG. 12B) Quercetin (QU, ▪), chrysin (CH, ▴), and DEAB (●). Data points are the mean±SEM. Best fit lines were calculated with GRAPHPAD PRISM.

### DETAILED DESCRIPTION OF THE INVENTION

The present invention relates from the identification of two physiological targets for CQ's that explain their actions as anti-malarial drugs and their side effects.

The present invention results from the demonstration that ALDH classes 1, 2 and 3 and QR2 are selective targets for existing CQ-based anti-malarial drugs, including CQ itself, PQ and MQ. The invention provides methods for identifying compounds that can be used to modulate the effects of ALDH and QR2 in vivo. Compounds so identified can be used as anti-malarial agents in the treatment of both CQ-resistant and CQ-nonresistant malaria. Such compounds can also be used in the treatment of autoimmune diseases, including lupus and arthritis (e.g., rheumatoid arthritis), as well as other medical indications susceptible to treatment using CQ like drugs (e.g., HIV).

The finding that quinoline containing antimalarials selectively inhibit ALDH indicates that other drugs that inhibit this enzyme can be expected to have utility in the treatment of diseases showing efficacy with CQ's. Antibuse (disulfiram) is one such drug. This is a known ALDH inhibitor and has been used for many years to treat alcoholism. The active metabolites of Antibuse (S-methyl-N,N-diethylthiocarbamoyl sulfoxide—MeDTC-SO) act as a pseudosubstrates and are competitive inhibitors of ALDH and alcohol dehydrogenase. Antibuse and other ALDH substrate-binding site inhibitors, can, therefore, mimic the actions of CQ-related drugs by acting at the active site on ALDH. Available data indicate that the CQ-related drugs act at the co-factor binding site. As inhibition of ALDH activity infers antimalarial activity, then drugs that act either at its substrate binding site (active site) or co-factor binding site can be expected to have antimalarial activity and can be expected to have utility in the treatment of other diseases showing efficacy with CQ's. (In cell-based assays of infection, Antibuse and other ALDH inhibitors (e.g., dethylamino butyric acid—DEAB) have been shown to have antimalarial activity.)

In addition, the invention further relates to the use of CQ-based drugs, and other ALDH inhibitors (including Antibuse) identifiable using the methods described herein, in the production of stem cells (e.g., human stem cells), for example, by blocking early differentiation without affecting proliferation.

In one embodiment, the present invention relates to methods of screening compounds for their ability to bind QR2 or ALDH and thereby to function, potentially, as anti-malarial agents as well as to function as therapeutics in other medical indications amenable to treatment with CQ-based drugs. The entire ALDH or QR2 molecule can be used in such binding assays or portions thereof can be used, for example, the nucleotide binding domain of ALDH or QR2 (e.g., residues 50-385 of ALDH), as can a fusion protein comprising ALDH or QR2 or portion thereof. Advantageously, portions of ALDH or QR2, or fusion protein containing same, include the enzyme active site and/or cofactor binding site.

Binding assays of this embodiment invention include cell-free assays in which ALDH or QR2, or portion thereof (or fusion protein containing same), is incubated with a test compound (proteinaceous or non-proteinaceous) which, advantageously, bears a detectable label (e.g., a radioactive or fluorescent label). Following incubation, the ALDH or QR2, or portion thereof (or fusion protein containing same), free or bound to test compound, can be separated from unbound test compound using any of a variety of techniques (for example, the ALDH or QR2, or portion thereof (or fusion protein containing same) can be bound to a solid support (e.g., a plate or a column) and washed free of unbound test compound. The amount of test compound bound to ALDH or QR2, or portion thereof (or fusion protein containing same), can then determined, for example, using a technique appropriate for detecting the label used (e.g., liquid scintillation counting and gamma counting in the case of a radiolabelled test compound or by fluorometric analysis).

Binding assays of this embodiment can also take the form of cell-free competition binding assays. In such an assay, ALDH or QR2, or portion thereof (or fusion protein containing same) (or anti-idiotypic antibody to the co-factor binding site or active site or peptide mimetic of the cofactor binding or active site), is incubated with a compound known to interact with ALDH or QR2 (e.g., a compound known to interact with the ALDH or QR2 cofactor binding site or ALDH or QR2 active site) (e.g., CQ, MQ, PQ, or ATP resin or other purine analog, pyrimidine or nicotinamide like nucleotide or anti-ALDH or anti-QR2 antibodies), which compound, advantageously, bears a detectable label (e.g., a radioactive or fluorescent label). A test compound (proteinaceous or non-proteinaceous) is added to the reaction and assayed for its ability to compete with the known (labeled) compound for binding to ALDH or QR2, or portion thereof (or fusion protein containing same). Free known (labeled) compound can be separated from bound known compound, and the amount of bound known compound determined to assess the ability of the test compound to compete. This assay can be formatted so as to facilitate screening of large numbers of test compounds, for example, by linking the ALDH or QR2, or portion thereof (or fusion protein containing same), to a solid support so that it can be readily washed free of unbound reactants.

ALDH or QR2, or portion thereof (or fusion protein containing same), suitable for use in the cell-free assays described above can be isolated from natural sources or prepared recombinantly or chemically. The ALDH or QR2, or portion thereof, can be prepared as a fusion protein using, for example, known recombinant techniques. Preferred fusion proteins can include as the non-ALDH or non-QR2 moiety, for example, GST or a histidine or FLAG tag. The non-ALDH or -QR2 moiety can be present in the fusion protein N-terminal or C-terminal to the ALDH or QR2 sequence.

As indicated above, the ALDH or QR2, or portion thereof (or fusion protein containing same), can be present linked to a solid support, including a plastic or glass plate or bead, a chromatographic resin such as Sepharose, agarose or cellulose, a filter or a membrane. Methods of attachment of proteins to such supports are well known in the art and include direct chemical attachment and attachment via a binding pair (e.g., biotin and avidin or biotin and streptavidin). It will also be appreciated that, whether free or bound to a solid support, the ALDH or QR2, or portion thereof (or fusion protein containing same), can be unlabeled or can bear a detectable label (e.g., a fluorescent or radioactive label).

Binding assays of the invention also include cell-based assays in which ALDH or QR2, or portion thereof (or fusion protein containing same), is present in a cell. Such assays can be conducted, for example, by introducing into a cell a substrate, that bears a detectable label, that is metabolized by ALDH or QR2 (or active portion thereof or fusion protein containing same) present in the cell to produce a detectable product. BODIPY, which is metabolizable by ALDH, is an example of one such substrate. The cell can then be contacted with the test compound and the effect of the test compound on the production of the detectable product monitored, a reduction of the production of detectable product in the presence of the test compound being indicative of an inhibitor of ALDH or QR2 (as appropriate, given the nature of the substrate).

To determine the specific effect of any particular test compound selected on the basis of its ability to bind ALDH or QR2, or portion thereof (or fusion protein containing same) (or inhibit (competitively or non-competitively) binding of a known ligand to ALDH or QR2), assays can be conducted to determine, for example, the effect of various concentrations of the selected test compound on K_{M}, Vₘₐₓ or specific activity. Assays can be conducted, for example, to determine the effect of a test compound on ALDH or QR2 activity using appropriate standard enzyme assay protocols.

In another embodiment, the invention relates to compounds identifiable using the above-described assays as being capable of binding to ALDH or QR2, or portion thereof (or fusion protein containing same) (or inhibiting (competitively or non-competitively) binding of a known ligand to ALDH or QR2), and/or modulating ALDH or QR2 activity. Such compounds can include novel small molecules (e.g., organic compounds) and novel polypeptides or proteins (including antibodies) or oligonucleotides. Compounds that inhibit ALDH or QR2 activity can be used as anti-malarial agents in the treatment of both CQ-resistant and CQ-nonresistant malaria. Such compounds can also be used in the treatment of autoimmune diseases, including arthritis (e.g., rheumatoid arthritis) and lupus, as well as other diseases/disorders known to be amenable to treatment with CQ like drugs. In addition, the compounds identified as ALDH inhibitors can be used in the production of stem cells (e.g., human stem cells), for example, by blocking early differentiation without affecting proliferation (known CQ-based drugs can also be used in stem cell production, e.g., Q, MQ, PQ or 4-aminoquinoline, as can Antibuse). Such ALDH inhibitors can be used under standard culture conditions to block retinoic acid production.

The compounds identifiable in accordance with the above assays can be formulated as pharmaceutical compositions. Such compositions can comprise the compound and a pharmaceutically acceptable diluent or carrier. The compound can be present in dosage unit form (e.g., as a tablet or capsule) or as a solution, preferably sterile, particularly when administration by injection is anticipated. The compound can also be present as a cream, gel or ointment, for example, when topical administration is preferred. The dose and dosage regimen will vary, for example, with the patient, the compound and the effect sought. Optimum doses and regimens can be determined readily by one skilled in the art.

When used in the production of stem cells, the concentration of ALDH inhibitor to be included in stem cell culture medium can be in the nanomolar to millimolar range, preferably in the micromolar range.

In yet a further embodiment, the invention relates to kits, for example, kits suitable for conducting assays described herein. Such kits can include ALDH or QR2, or portions thereof, or fusion proteins comprising same. These components can bear a detectable label. The kit can include such components disposed within one or more container means. The kit can further include ancillary reagents (e.g., buffers) for use in the assays.

When administered at therapeutic levels, CQ's are known to actively accumulate, often at [mM] at sites that produce both therapeutic as well as untoward effects, namely the infected RBC's, skin and eye (Linquist, Acta Radiol. Diagn. (Stockh) 325:1-92 (1973)). Interestingly, these tissues also contain some of the highest concentrations of ALDH in the human body (Linquist, Acta Radiol. Diagn. (Stockh) 325:1-92 (1973)), a fact that may provide mechanistic insight as to why CQ's become concentrated in certain cell types and not others. For example, it has long been known that there is a strong correlation between CQ distribution and the skin pigment melanin (for review see International Human Genome Sequencing Consortium, Nature 409:860-921 (2001)). Melanin is found in pigmented tissues, such as the skin and retina. ALDH1 is also highly expressed in these tissues. Given the high levels of ALDH1 in RBC, skin and the retina, ALDH1 may provide a mechanism for the concentration of the drug in these locations. Inhibition of ALDH by CQ's in the RBC may contribute anti-malarial effects by reducing endogenous NADH levels. One of the functions of ALDH is to generate intracellular NADH by oxidation of aldehydes. If ALDH contributes significantly to intracellular NADH levels, inhibition of its activity is likely to compromise the ability of the cell to reduce methemoglobin to Hb. This task is normally accomplished through the actions of the NADH dependent enzyme methemoglobin reductase. Importantly, the major side effects of CQ's, photosensitivity (skin) and retinopathy (eye) can now also be explained by inhibition of ALDH. In the eye, ALDH has two primary functions, to oxidize aldehydes that may be formed from harmful ultraviolet radiation, and to generate retinoic acid (visual pigment) from retinaldehyde (Vitamin A). The retinopathy associated with the use of high doses of CQ in the treatment of arthritis and lupus (10 times that used to normally treat malaria) can therefore be explained by an inhibition of ALDH. Accumulation of retinaldehyde in visual tissues is associated with the pathology that develops in patients-treated with high [CQ] which supports this hypothesis (Beek et al, Dermatol. 19:1-11 (1971)), Rynes, Brit. J. Rheumatol 36:799-805 (1997), Lindquist, Acta Radiol. Diagn. (Stockh) 325:1-92 (1973)). Recently it has been demonstrated that the high expression of ALDH (comprising 15% of soluble protein) in rabbit keratocytes contributes to corneal transparency (Jester et al, J. Cell Sci. 112:613-622 (1999)), and that ALDH is highly expressed in human corneas (King et al, J. Exp. Zool. 282:12-17 (1998))). These reports coupled with the observation that CQ can induce keratopathy, presumably due to the deposition of the drug in the cornea, is a further indication of a role for CQ binding to ALDH in the induction of side effects of the drug.

The findings presented herein do not directly explain the mechanisms by which malaria has become resistant to CQ's. However they do indicate that this resistance has almost certainly arisen to enable the organism to overcome the oxidative stress that is induced in RBC's by the CQ's. Potential mechanisms of resistance have been identified at least at the genetic level, in cultures of resistant strains of P. falciparum and other malarial species (Miller, Nature 383:480-481 (1996), Foote et al, Nature 345:255-258 (1990), Wellems et al, Nature 345:253-255 (1990), Su et al, Cell 91:593-603 (1997)). A primary candidate is the multi drug resistance transporter that acts presumably to pump CQ out of the infected cell (Miller, Nature 383:480-481 (1996), Foote et al, Nature 345:255-258 (1990)). The findings described herein, however, point to two new protein targets for new anti-malarial drug discovery. First, the selectivity of CQ's for QR2 and ALDH along with their limited tissue expression indicate that the usefulness of these drugs can be extended, for example, through combinatorial approaches to find more selective inhibitors, thus making these enzymes attractive targets for the identification of new anti-malarials. If mutations in multi drug resistance transporters in P. falciparum lead to CQ resistance, then clearly a new structurally different QR2 or ALDH inhibitor is likely to overcome such resistance. Rational drug design approaches can be used, where appropriate, to identify new generations of drugs that can be used to treat autoimmune diseases (e.g., rheumatoid arthritis and lupus), as well as other medical indications susceptible to treatment with CQ like drugs, and that lack side effects by eliminating binding to ALDH. The mechanisms for CQ's effectiveness in alleviating the symptoms of these diseases are not known. Data provided herein indicate that ALDH and QR2 are the only classes of enzymes that will bind the drug in vivo.

Certain aspects of the invention can be described in greater detail in the non-limiting Example that follows.

### EXAMPLE 1

Experimental Details

Plasmodium falciparum 3D7 strain was obtained from the MR4/ATCC. Parasites were grown at a 2% hematocrit in type O+ blood (Valley Biomedical) and harvested as described (Schlichtherle et al (eds) Methods in Malaria Research 77(MR4/ATCC, Manasas, Va. 2000).

ATP-affinity array chromatography. In whole mouse experiments, the animal (except for its tail, feet, skin and intestines) was frozen in liquid N₂ and blended in buffer A containing 50 mM Hepes, pH 7.5, 50 mM NaCl, 10 mM MgCl₂, 0.1% NONIDET- P40, 1 mM dithiothreitol, 1 μg/ml leupeptin, 100 μg/ml pefabloc and 10 μg/ml aprotinin. Mouse lysate was clarified by centrifugation for 1 hr at 100,000×g. For infected and non-infected RBC's˜1.5×10⁹ cells were mixed with an equal volume of 2×buffer A, rocked for 30 min at 4° C., and clarified by centrifugation for 1 hr at 100,000×g. Supernatants were dialyzed overnight against 50 mM Hepes, pH 7.5, 50 mM NaCl, 10 mM MgCl₂ and 1 mM dithiothreitol. Following dialysis, mouse or RBC lysate was applied to ATP affinity arrays previously equilibrated in buffer A. The arrays were washed with ˜50 volumes of buffer A, followed by buffer A containing 1 M NaCl, and then re-equilibrated in buffer A. For elutions, all compounds were dissolved in buffer A and adjusted to pH 7.5. Proteins were resolved by 12% SDS-PAGE and visualized by staining either with Coomassie brilliant Blue R-250 or silver nitrate. Alternatively, proteins were transferred to membrane GeneMate (Kaysville, Utah) in transfer buffer (25 mM Tris, 200 mM glycine, 20% methanol, and 0.01% SDS) for mixed peptide sequencing (Damer et al, J. Biol. Chem. 273:24396 (1998)). hALDH 1 and hQR2 purification and activity assays. Fresh human red blood cells (RBC) were obtained from Valley Biomedical, washed twice with PBS, and lysed in buffer B (0.1% Triton X, 50 mM Tris pH 7.5, 125 mM NaCl, 1 μg/mL aprotonin, 1 μg/mL pepstatin and 10 μg/mL PEFABLOC). Lysates were centrifuged twice at 125,000×g for 1 hr at 4° C. Cleared supernatant was filtered through a 0.22 μM filter and loaded onto PQ-Sepharose. Pure hALDH was eluted with 5 mM NADH and pure hQR2 with 5 mM NMeN. The eluants were dialyzed overnight at 4° C. against 25 mM Tris pH 8.0 and 1 mM DTT. hALDHl was assayed as described in Table 2. Purified hQR2 was assayed at 22° C. in 100 μM N-methyldihydronicotinamide, 30 μM menadione, 0.1% Triton X-100, 50 mM Tris pH 8.5, 100 μM dicoumarol (unless stated otherwise). N-methyldihydronicotinamide was synthesized (Ortiz-Maldonado et al, Biochemistry 38(50):16636-16647 (1999)) and stored in 100 mM Tris-sulfate pH 8.0.

Primaquine-Sepharose preparation. PQ-Sepharose was prepared by mixing primaquine diphosphate with NHS-activated Sepharose 4 Fast Flow (Pharmacia) in 100 mM Hepes, pH 8.3, for ˜12 hrs at 23° C. Following coupling, the resin was washed extensively to remove unbound primaquine and non-reacted groups blocked with 1 M Tris-HCl, pH 8.0.

Protein sequencing. Edman based mixed peptide sequencing was carried out as described (Cynthia et al, J. Biol. Chem. 273:24396 (1998)). For mass spectrometry, protein samples were in-gel digested with trypsin according to the method of (Shevchenko et al, Anal. Chem. 68:850-858 (1998)). Extracted tryptic peptides were purified with Poros R2 (PerSeptive Biosystems, Framingham, Mass.) according to a protocol on the website http://www.protana.com. The extracted peptides were concentrated in a nanoES capillary and placed in the source head of an API QSTAR Pulsar Hybrid mass spectrometer. Peptide ions of charge state (+2) or (+3) were selected for CID using nitrogen collision gas. Mass spectra data were initially analyzed with Q-analyst software (AB, Foster city, Calif.) and searches performed against a non-redundant sequence database (nrdb). Peptide sequences derived from this analysis were used in FASTS or TFASTS to determine the statistical significance of all protein identifications (Damer et al, J. Biol. Chem. 273:24396 (1998)).

Results

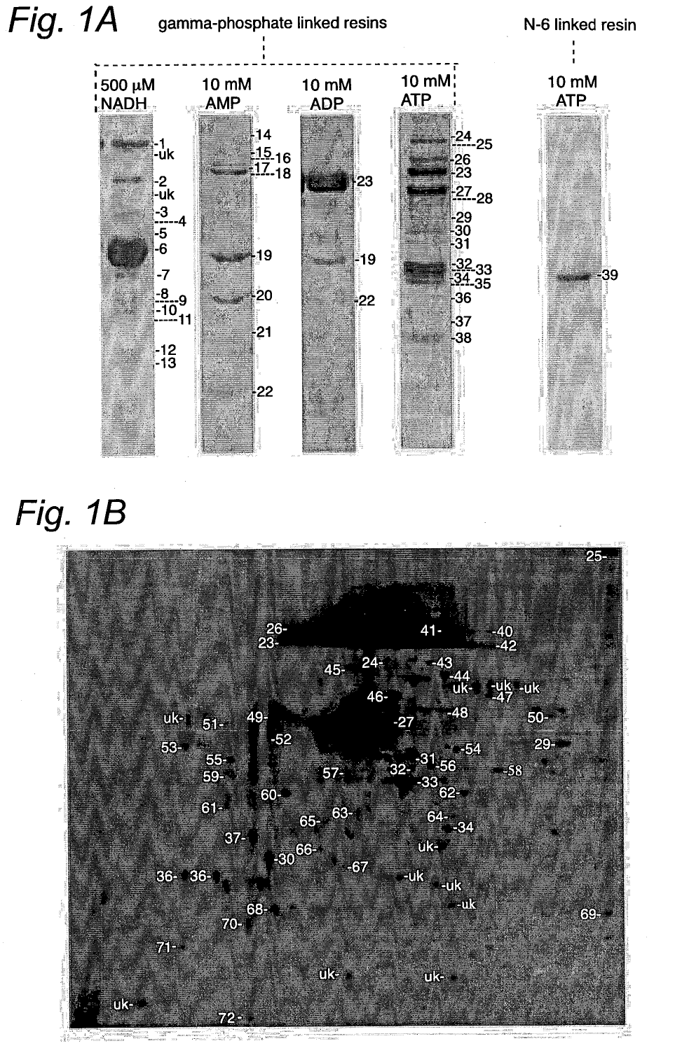

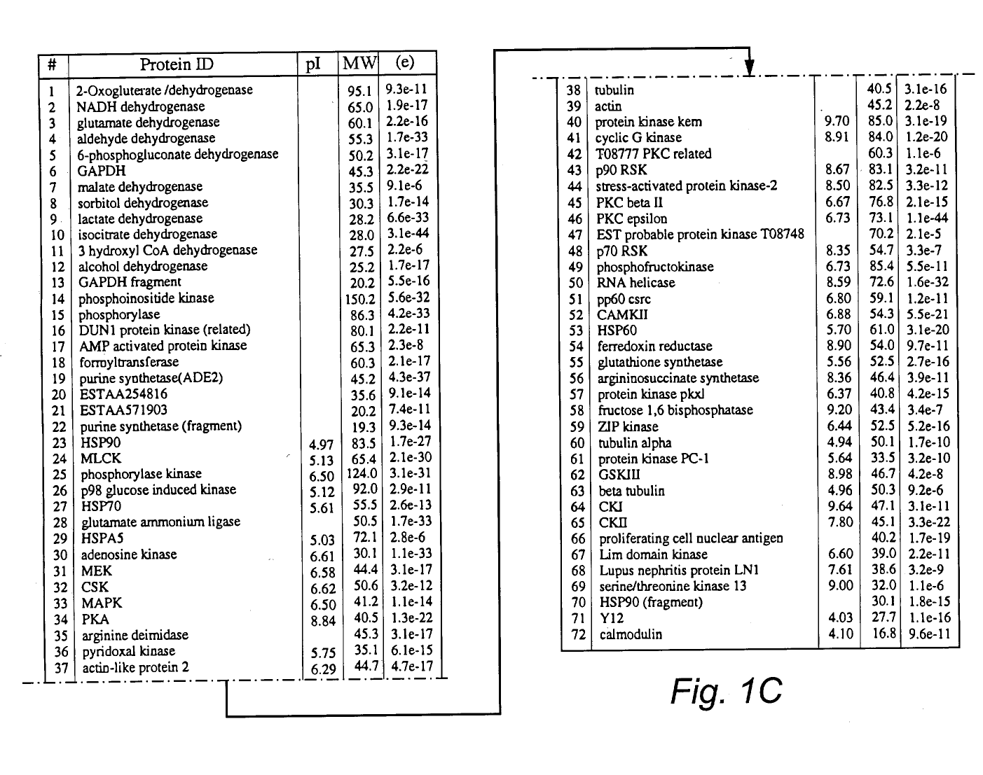

Capture of the mouse, RBC and P. falciparum purine binding proteomes. Purine containing nucleotides have multifunctional roles in many aspects of human and malarial biology. They form the precursors of RNA and DNA, function as regulators of allosteric enzymes, mediate both extracellular and intracellular signals, participate in dehydrogenase reactions in the form of NAD⁺ and NADP⁺, and serve as substrates for both protein and non-protein kinases (Colman, Ann. Rev. Biochem. 52:67-91 (1983), Knighton et al, Science 253(5018):407-414 (1991)). To capture the purine binding proteome, gamma-phosphate linked ATP-resin was synthesized (Haystead et al, J. Biochem. 214(2):459-467 (1993)). This resin has been used to purify protein kinases from tissue extracts as well as identify a new target, ADE2, for the HSP90 binding drug geldanamycin (Haystead et al, Current Drug Discovery 1:22-24 (2001)). The resin has been further developed into a parallel affinity array apparatus for screening small molecule libraries for competitive inhibitors of purine binding proteins. In the present study, this resin was used to identify quinoline antimalarial binding proteins from the entire purine binding proteome of the mouse and human RBC infected with P. falciparum asexual blood stage parasites. To test selectivity, the ATP resin was first charged with whole mouse extract. After washing with salt to remove non-specifically bound proteins, the resin was sequentially eluted with NADH, AMP, ADP and ATP. Proteins in the nucleotide eluates were characterized by 1D or 2D SDS-PAGE and silver staining (FIG. 1). On average, ˜400 distinct proteins (N=8) were detected in the gels. Of these, ˜100 proteins of varying abundance were immediately accessible to protein sequencing either by mixed peptide sequencing (Damer et al, J. Biol. Chem. 273:24396 (1998)) or mass spectrometry. Using the T/FASTF/S algorithms (Damer et al, J. Biol. Chem. 273:24396 (1998), without exception, the sequenced proteins were identified in the public data bases as purine binding proteins, demonstrating the selectivity of the ATP resin for this class of enzymes. Analysis of PTH amino acid recovery during mixed peptide sequencing showed proteins of high and low cell copy number were recovered by the resin (e.g. GAPDH 2.5 nmol ±0.41 nM n=8 SDM; MAPK 0.7±0.15 pmol n=8 SDM; GSKIII 0.2±0.15 pmol n=4 SDM). Importantly, dissociation constants for many of the identified proteins for purine containing nucleotides are generally between 10-50 μM, indicating that the differential recovery of individual proteins in the nucleotide washes was a reflection of cell copy number rather than differences in affinity between proteins for the immobilized nucleotide. Of the proteins that were sequenced, ˜70% of these were identified by FASTF or FASTS in the annotated protein databases. Four proteins were identified in the EST databases. Sequence homology searches with FASTA identified two of these, T08777 and T08748 as probable protein kinases. Mixed sequence data were obtained on nine proteins that were classified as unknown proteins because they were not identified by FASTF or TFASTF in the public databases at the time. Twelve proteins that were selected for sequencing were not identified because they were below the sequencing sensitivity (<10 fmol).

Inspection of the proteins listed in FIG. 1 shows that they either belong to the protein kinase, dehydrogenase, ATPase class's of purine binding proteins or are non-conventional purine binding proteins. Notably, the classical mononucleotide binding family members belonging to the motor protein family are absent. The crystal structures of several family members of the identified proteins have been solved (e.g., several of the dehydrogenases, protein kinases and heat shock proteins) with nucleotide bound and this explains the selective recovery of the three conventional class's, as well as the lack of recovery of classical mononucleotide binding family members (Rynes, Brit. J. Rheumatol. 36:799 (1997), Eventoff et al, CRC Crit. Rev. Biochem. 3(2):111-140 (1975), Sprang et al, Science 254:1367-1372 (1991)). In the case of the latter family, the crystal structures of several member enzymes show that generally the phosphates of the bound nucleotides are oriented inward, away from the surface of the binding pockets (Rayment et al, Science 261:50-58 (1993)). This orientation would sterically disfavor interaction with gamma phosphate linked nucleotide. Importantly, when mouse extract was passed over ATP resin, in which the nucleotide was bound either through adenosine at N6 or the ribose at C5, few proteins were recovered (FIG. 1).

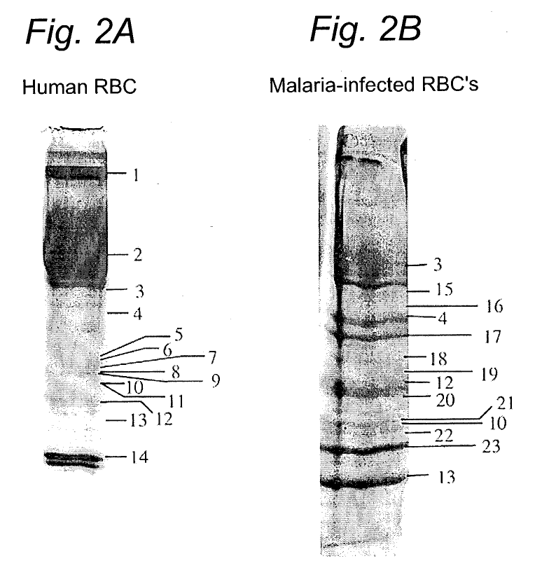

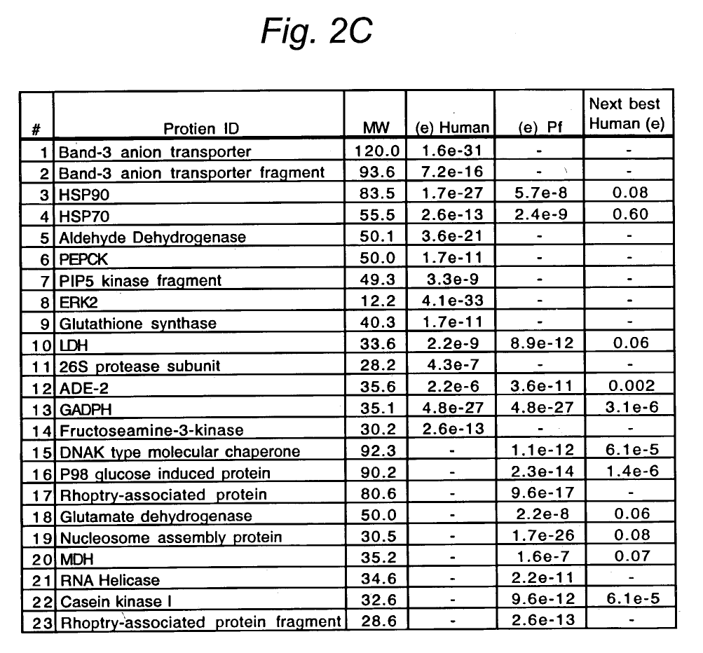

To test the selectivity in other species, RBC and P. falciparum infected RBC extracts were passed in parallel over the ATP resin in affinity array apparatus. Sufficient cell mass (10⁷-10⁸ cells) was applied to each channel in the array to ensure detection and recovery of proteins expressed at 100 copies/cell (1 fmol). A selection of the bound proteins from each channel were sequenced following their elution with SDS. FIG. 2 shows that without exception all of the eluted proteins were purine binding proteins. As with mouse, all protein identifications were made following searches of the data bases with multiple peptide sequences derived by mass spectrometry or by mixed peptide sequences using FASTF or FASTS (Damer et al, J. Biol. Chem. 273:24396 (1998)). This search strategy was important in the case of proteins isolated from infected cells, since these were a mixture of human and P. falciparum proteins. Using multiple peptide alignments, expectation (e) scores for top scoring P. falciparum proteins ranged from e⁻⁶ to e⁻³³ compared with their respective human homologs which generally ranged from 0.13 to e⁻¹⁴ (FIG. 2).

Results from protein sequencing experiments demonstrate that the ATP resin captured a significant portion of the mouse, human RBC and P. falciparum purine binding proteomes. Indeed, using the identified mouse sequences, a search of the completed human genome indicated that the captured proteome represents ˜4% of the expressed genome (Venter et al, Science 291:1304-1351 (2001), International Human Genome Sequencing Consortium, Nature 409:860-921 (2001)). Because of the numbers of proteins captured, and the finding that the majority of these proteins have similar affinities for their respective purine nucleotides, the trapped proteome represents an ideal matrix to test the selectivity of quinoline containing drugs and purine analogs.

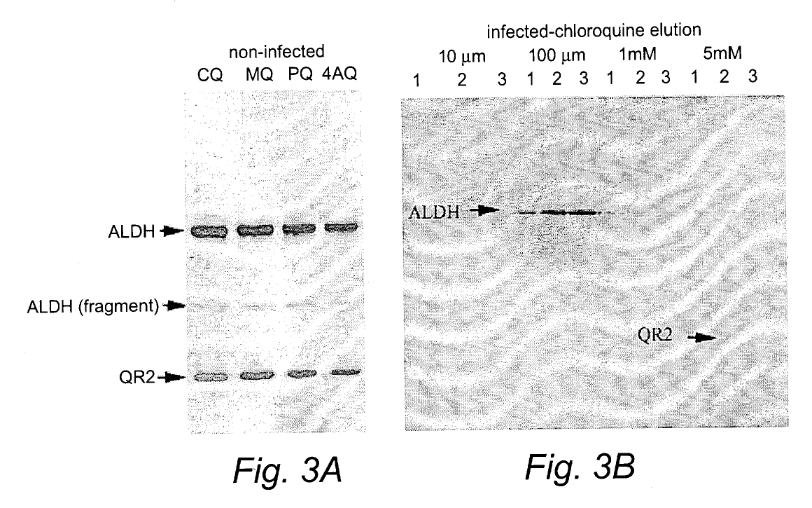

Testing the selectivity of CQ's against the human and malarial purine binding proteome. The structural similarities of CQ's with purine nucleotides suggested that these drugs may selectively interact with and inhibit proteins in the purine nucleotide binding proteome in vivo. An ATP affinity array was charged with RBC and P. falciparum (blood stage) infected RBC extract. Each array channel was washed and then eluted in parallel with CQ, MQ, PQ and 4 amino quinaldine (4AQ). FIG. 3 shows that in the case of non-infected red cells, all four tested drugs selectively eluted two proteins from the array at [mM]. Peptide sequencing by mass spectrometry and data base searches with FASTS identified these proteins as human aldehyde dehydrogenase type 1 (hALDHl) and quinone reductase type 2 (hQR2) (Table 1). Given the number of other purine binding proteins captured by the array, these data suggest that all four drugs are highly selective towards hALDH1 and hQR2 in non-infected red cells. When the ATP affinity array charged with infected red cells was eluted with increasing concentrations of CQ, two proteins were also selectively eluted (FIG. 3). Surprisingly, microsequencing by mass spectrometry identified these proteins as hALDH1 (1e⁻³⁰) and hQR2 (8.1e⁻³⁴), respectively. Notably, the abundance of both proteins was strikingly reduced in the parasitized cells, presumably because of digestion of the host proteins by the parasite. A search of the complete P. falciparum genome with FASTS or TFASTS using multiple peptide sequences derived from these proteins or the full length human sequences did not identify a parasite homolog. These findings suggest that any actions of CQ's on the infected cell purine binding proteome (includes human and P. falciparum) is through enzymes produced by the human genome and not by P.falciparum. Importantly, dapsone, an antimalarial drug of the sulfur class whose mechanism of action is unrelated to the quinolines (inhibits de novo folate biosynthesis) did not elute either hQR2, hALDH1 or any other protein from the ATP affinity array charged with infected or non infected red cell extract.

TABLE 1

Identification of hALDH1 and hQR2 by FASTS. Protein bands were
excised from silver stained gels and digested with trypsin. The tryptic
peptides were extracted and analyzed by nano spray in an ABI QSTAR-
pulsar mass spectrometer. The indicated amino acid sequences were used
to search the HCBI/Blast NR data base using the FASTS algorithm.

| | | |
|---|---|---|
| | Tryp- | |
| | tic | |
| | Pep- | |
| | tide | |
| | Mass | Protein |
| FASTS Alignment | Da | (e) |

| | | | |
|---|---|---|---|
| >DEHUE1 | 1- 41:--------------------------------------------------- : | | |
| QUERY | TIPIDGNFFTYTR--------------------------- | 772.79 | |
| | ::::::::::::: | | |
| DEHUE1 | ATMESMNGGKLYSNAYINDLAGCIKTLRYCAGWAUKIQGRTIPIDGNFFTYTRHEPIGVCGQIIPWNFPLVMLIWKIGPA | | hALDH |
| | 110 120 130 140 150 160 170 180 | | Class 1 |
| QUERY | -------------------------------------------------------------------------------- | | |
| DEHUE1 | LSCGNTVVVKPAEQTPLTALHVASLIKEAGFPPGVVNIVPGYGPTAGAAISSHMDIDKVAFTGSTEVGKLIKEAAGKSNL | | 39e−2.6 |
| | 190 200 210 220 230 240 250 260 | | |
| QUERY | ---------------------------------------------LTVEESTYDEFVR---------------------- | 823.38 | |
| | :::::::.:::: | | |
| DEHUE1 | KRVTLELGGKSPCIVLADADLONAVEFAHHGVIFYIIQGQCCIAASRIFVFESIYDFVRIVERARKYILGNPLTPGVTQG | | |
| | 270 280 290 300 310 320 330 340 | | |
| QUERY | -----------------------------------------------------------------------------ANN | 795.37 | |
| DEHUE1 | PQIDKEQYDKILDLIESGKKEGAKLECGGGPWGNKGYFVQPTVFSNVTDEMRIAKEEIFGPVQQIMKFKSLDDVIKRANN | | |
| | 350 360 370 380 390 401 410 420 | | |
| QUERY | TFYGLSAGVFTK | | |
| | :::::::::::: | | |
| DEHUE1 | TFYGLSAGVFTKDIDKAITISSALQAGTVWVNCYGVVSAQCPFGGFKMSGNGRELGEYGFHEYTEVKTVTVKISQKNS* | | |
| | 430 440 450 460 470 480 490 500 | | |
| >gi\|991 | 1- 72:--------------------------------------------------------------------- | | |
| QUERY | VLIVYAHQEPK-------NVAVDELSR-----------------------------------------------S | 648.00 | |
| | ::::::::::: ::::::::: : | 501.00 | hQR2 |
| gi\|991 | MAGKKVLIVYAI1QEPKSFNGSIKNVAVDEISRQGCTVVSDIYAINFEPRATDKTILSNPIWFNYGVETIIEAYKQRS | | |
| QUERY | LASDITDEQKK--------------------------------------------------------------------- | 667.00 | |
| | ::::::::::: | | 7.8e−106 |
| gi\|991 | LASDITDEQKKREADLVIFQFPLYWFSVPAILKGWMDRVLCQGFAFDIPGFYDSGLLQGKLALLSVTTGGTAEMYTKTG | | |
| | 90 100 110 120 130 140 150 160 | | |
| QUERY | -----------------------VLAPQISFAPEIASEEER | 994.00 | |
| | :::::::::::::::::: | | |
| gi\|991 | VNGDSRYFLWPLQHGTLHFCGFKVLAPQISFAPEIASEEERGMVAAWSQRLQTIWKEEPIPCTAHWHFGQ | | |
| | 170 180 190 200 210 220 230 | | |

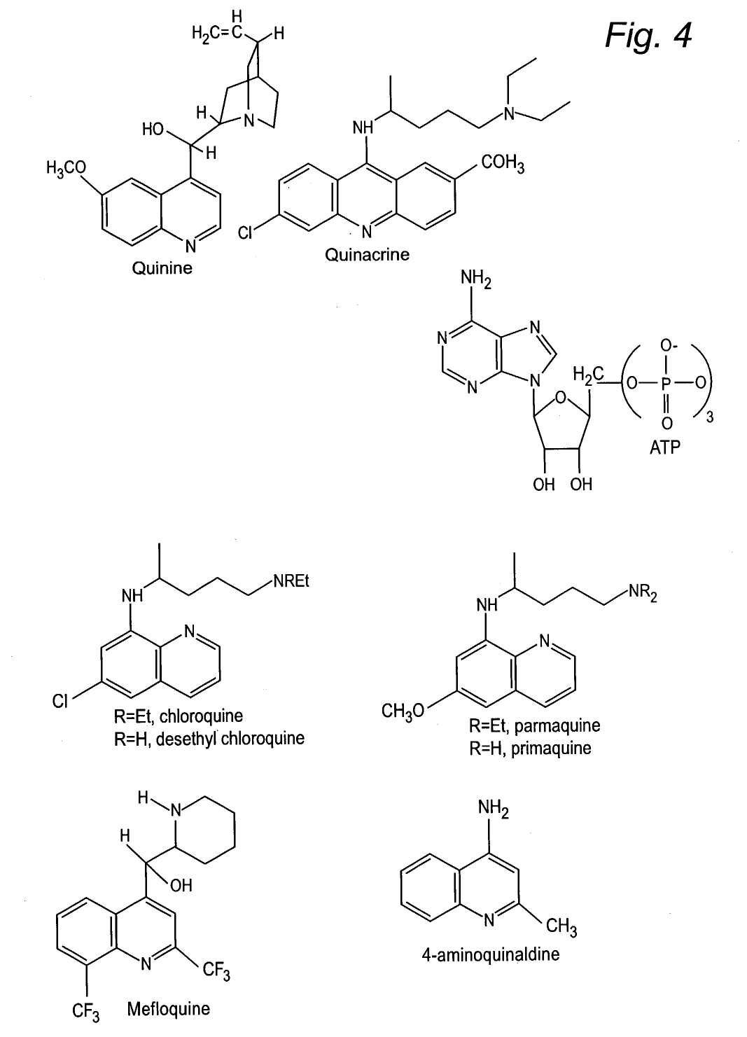

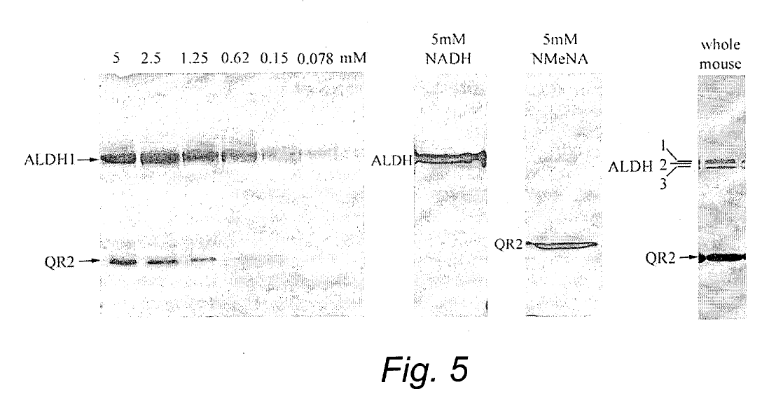

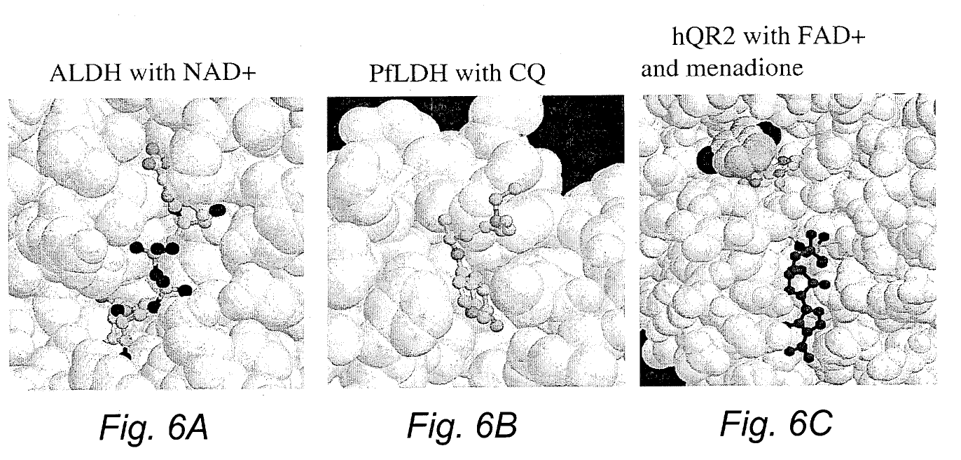

The effects of CQ's on hALDH1 and hQR2. Examination of the structures of all four drugs tested shows they share structural similarity in their quinoline rings, with greatest variation at the C4 (CQ, MQ and 4AQ) and C8 (PQ) positions (FIG. 4). Binding of these compounds to hALDH1 and hQR2 is therefore likely to be through their quinoline ring moieties rather than through additions at the C4 and C9 positions. This hypothesis is supported by the finding that 4AQ is as equally effective at eluting hQR2 and hALDH1 as the other three derivatives. Specific interaction of hALDH1 and hQR2 with the quinoline rings was confirmed using PQ-Sepharose. PQ was immobilized through its primary amine and RBC extracts passed over the resin. FIG. 5 shows that hALDH1 and hQR2 were the only two proteins recovered from the resin following elution with increasing [PQ]. Similar results were obtained if the column was eluted with CQ, MQ or 4AQ. The 3D structures of human hALDH1 and hQR2 with bound nucleotides and substrate provide clues to the mechanism of recovery on the ATP affinity array and binding to CQ's (FIG. 6). FIG. 6A indicates that recovery of hALDH1 is likely due to interaction of the immobilized ATP with the adenosine and phosphate binding pockets normally occupied by NAD+. Elution from the array with CQ's is therefore due to competition with the adenosine binding pocket of NAD+. Evidence in support of this hypothesis was obtained following selective elution of hALDH1 from PQ-Sepharose with NAD⁺ (FIG. 5). The 3D structure of the structurally related PfLDH with CQ bound further eludes to the mechanism of drug interaction with hALDH1 (FIG. 6B). FIG. 6B shows the quinoline portion of the drug buried within the adenine binding pocket of NADH, while the C4 dibasic hydrophobic tail solvent accessible at the surface. Interaction of PQ, and hence other CQ's, with HALDH1 is therefore also likely to be through the adenosine binding pocket. Examination of the 3D structure of hQR2, shows that either the adenosine binding pocket of FAD+ or the quinone substrate binding pocket could explain the recovery and elution of hQR2 from the ATP affinity array (FIG. 6C). Both pockets are also potential targets for CQ. To test which pocket was the primary site of interaction, PQ-Sepharose was charged with whole RBC extract and eluted with FAD+, the substrate analog n-methyldihydronicotinamide (NMeNA) or menadione (FIG. 5). FIG. 5 shows that hQR2 was the only protein eluted with NMeNA, suggesting the quinone substrate binding pocket is both the site of interaction with the immobilized ATP as well as the site of action of CQ's. In stark contrast, neither hALDH1 or hQR2 was selectively eluted with FAD+ (FIG. 5). As a final test of selectivity of the CQ's, whole mouse extract was applied to PQ-Sepharose. Remarkably, only QR2 and the 3 known isoforms of ALDH were recovered following elution of the resin with CQ (FIG. 5).

To test the hypothesis that hALDH1 and hQR2 are inhibited by CQ's, both enzymes were purified from human RBC extracts using PQ affinity chromatography and tested in inhibition assays (Table 2). Table 2 shows that hQR2 is potently inhibited at [μM] with the tested drugs. Importantly, purified hQR1, which is 49% identical to QR2, was not inhibited by any of the tested drugs (Table 2). Because of the co-absorbance of NADH and CQ's at 340 nm, an HPLC based assay was developed to determine the effects of these drugs on hALDH1 activity. hALDH1 clearly binds CQ and its analogs under our assay conditions; however, CQ was found to be a relatively weak inhibitor of hALDH1 activity in the presence of physiological [NADH] (Table 2).

Table 2. CQ and its derivatives inhibit hQR2 and hALDH1 activity in vitro. Purified hQR2 (K_{cat}=1.3 μmol⁻¹min⁻¹ mg¹), hQR1 (K_{cat}=16 μmol⁻¹min⁻¹ mg⁻¹) and hALDH1 (K_{cat}=4.16 μmol⁻¹min⁻¹ mg⁻¹) were assayed as described in the methods.

For QR2 Kd apparent values determined using the expression

$$Ki = \frac{( Ao - {A}_{app} )}{{A}_{app}} ( 1 + \frac{1}{{K}_{M}} [ M ] )$$

The Kd was determined as

$$Kd = \frac{1}{Ki}$$

Where Aₒ=rate in the absence of drug; Aₐₚₚ=rate+drug; [M]=concentration of menadione.

Values for the hQR2 Km for menadione were 14 μM, the assay used 20 μM.

Values for hALDH1 were determined by HPLC assay. Purified hALDH1 activity was assayed in the presence absence of 0.5 mM CQ. Products of the reaction were separated with a gradient of acetonitrile in 10 mM triethylamine acetic acid. CQ and NADH peaks were identified by their signature spectra using an online photodiode array detector.

QR1 preparation and activity assay: QR1 was prepared from 10 g rabbit liver according to Sharma et al (Nature 376:380 (1995)) and Goldberg et al (Proc. Natl. Acad. Sci. 87:2931 (1990)). Purified QR1 was ˜95% homogeneous on silver stained gels and its activity was inhibited 100% upon addition of 5 μM dicumarol. QR1 activity was assayed spectrophometrically by monitoring absorbance at 340 nM. Reaction mixture included: 20 μM menadione, 50 μM NADH, 50 mM Tris pH 7.5 and 0.1% Triton x-100. [CQ] tested was 1 mM, the experiment was repeated several times with similar results.

| Experiment | Activity |
|---|---|
| QR2 + CQ | Kd₍ₐₚₚ₎ = 24 ± 10 μM |
| QR2 + PQ | Kd₍ₐₚₚ₎ = 9.3 ± 3.2 μM |
| QR2 + 4AQ | Kd₍ₐₚₚ₎ = 0.81 ± 0.2 μM |
| ALDH alone | 48.42 ± 9.3 pmol |
| ALDH + CQ | 26.62 ± 6.1 pmol |
| QR1 alone | 15.95 μmol⁻¹min⁻¹μg⁻¹ |
| QR1 + CQ | 16.75 μmol⁻¹min⁻¹μg⁻¹ |

### EXAMPLE 2

Experimental Details

Plasmodium cultures. Plasmodium falciparum strain 3D7 was obtained from the MR4/ATCC and grown according to the included specifications. Parasites were harvested by saponin lysis as previously described (Schlichtherle et al (eds) Methods in Malaria Research 77(MR4/ATCC, Manasas, Va. 2000). P. falciparum growth was measured by (³H)hypoxanthine uptake as previously described (Schlichtherle et al (eds) Methods in Malaria Research 77(MR4/ATCC, Manasas, Va. 2000).

Reagents. All compounds were obtained from Sigma-Aldrich (St. Louis, Mo.) except for Mefloquine-HCl obtained from Hoffmann-La Roche (Basel, Switzerland).

Preparation of ATP, PQ and HCQ-Sepharose. ATP-Sepharose was prepared as previously described (Haystead, Eur. J. Biochem. 2142):459-467 (1993)). PQ-Sepharose was prepared by coupling primaquine diphosphate to NHS-activated Sepharose 4 Fast Flow obtained from Pharmacia (Peapack, N.J.) in 100 mM Hepes, pH 8.3 for ˜12 hrs at room temperature. HCQ-Sepharose was prepared by coupling hydroxychloroquine to epoxy-activated Sepharose 6B (Pharmacia) according to the manufacturer's instructions.

ATP, PQ and HCQ-Sepharose affinity

chromatography. P. falciparum-infected or non-infected RBCs were lysed by mixing with an equal volume of 2×buffer A and rocking for 30 min at 4° C. 1×buffer A: 50 mM Hepes, pH 7.5, 50 mM NaCl, 10 mM MgCl₂, 0.1% NONIDET-P40, 1 mM dithiothreitol, 1 μg/ml leupeptin, 100 μg/ml pefabloc and 1 μg/ml aprotinin. For mouse homogenates, a whole mouse (except for the tail, feet, skin and intestines) was frozen in liquid N₂, crushed, and blended in buffer A. Mouse or RBC lysate was clarified by centrifugation for 1 hr at 100,000×g and applied to the ATP or quinoline drug affinity columns equilibrated in buffer A. The columns were washed with ˜100 column volumes of buffer A, followed by buffer A containing 1 M NaCl, and then re-equilibrated in buffer A. For elutions, all compounds were dissolved in buffer A and adjusted to pH 7.5. Proteins were resolved by 12% SDS-PAGE and visualized by staining with Coomassie brilliant blue R-250 or silver nitrate. Alternatively, proteins were transferred to PVM membrane (Kaysville, Utah) for mixed peptide sequencing (Damer et al, J. Biol. Chem. 273(38):24396-24405 (1998)).

Protein sequencing. Edman based mixed peptide sequencing was carried out as described (Damer et al, J. Biol. Chem. 273(38):24396-24405 (1998)). The mixed sequences were sorted and matched against the entire published protein (SWISS-PROT, NCBI or mouse EST) or DNA databases with the FASTF or TFASTF algorithms respectively (Damer et al, J. Biol. Chem. 273(38):24396-24405 (1998), Mackey et al, Molecular and Cellular Proteomics 1.2:139-147 (2002)). For mass spectrometry, protein samples were in-gel digested with trypsin according to the method of Shevchenko (Anal. Chem. 68(5):850-858 (1996)). Extracted tryptic peptides were purified with Poros R2 (PerSeptive Biosystems, Framingham, Mass.) according to a protocol on the website: http://protana.com. The extracted peptides were concentrated in a nanoES capillary and placed in the source head of an API QSTAR Pulsar Hybrid mass spectrometer. Mass spectra data were analyzed with Q-analyst software (AB, Foster city, Calif.) to derive de novo peptide sequences. Peptide sequences were searched against the nonredundant sequence database using FASTS (Mackey et al, Molecular and Cellular Proteomics 1.2:139-147 (2002)).

Purification of native ALDH1, QR2 and QR1. RBC extract was prepared as described above and applied to PQ-Sepharose equilibrated in buffer A. ALDH1 and QR2 were obtained by eluting the column with 5 mM b-NAD⁺ and NMeH respectively. QR1 was purified from rabbit liver as previously described (Lind et al, Methods Enzymol. 186:287-301 (1990)). All enzymes were sequenced to confirm their identify and were >90% pure as judged by SDS-PAGE and silver staining.

Cloning of human QR2. Human QR2 was PCR amplified from human liver cDNA (Clontech) with the following primers: 5′GCTATGGCAGGTAAGAAAGTACTC-3′ and 5′-GCCACAGAGTTATTGCCCGAAGTG-3′ and cloned into the pGEX-4T-2 GST expression vector (Pharmacia). GST-tagged QR2 was purified and the GST tag removed according to the manufacturers instructions.

ALDH1, QR2 and QR1 activity assays. ALDH1 activity was determined using an HPLC-based assay because of the co-absorbance of CQ and NADH at 340 nm. Reaction products were separated with a gradient of acetonitrile in 10 mM triethylamine acetic acid. CQ and NADH peaks were identified by their signature spectra using an online photodiode array detector. QR2 activity was assayed in triplicate with recombinant QR2 (at 96 ng/mL) by measuring the absorbance at 365 nm in a buffer containing 50 mM Tris-HCl, pH 8.5, 50 μM NMeH, 5-30 μM menadione, and 0.1% Triton X-100. NMeH was synthesized as previously described (Ortiz-Maldonado et al, Biochemistry 38(5):16636-16647 (1999)) (Ortiz-Maldonado et al, Biochemistry 38:16636-16647 (1999)). QR2 K₁ values were calculated using KinetAsyst II by fitting the experimental data to the equations of Cleland (Methods Enzymol. 63:103-138 (1979)). QR1 activity assays were performed in triplicate according to the method of Chen and colleagues (Chen et al, Mol. Pharmacol. 56(2):272-278 (1999)). QR1 IC₅₀ values were calculated using GraphPad Prism.

Results

Capture of the mouse, RBC and P. falciparum purine binding proteomes on ATP-Sepharose.

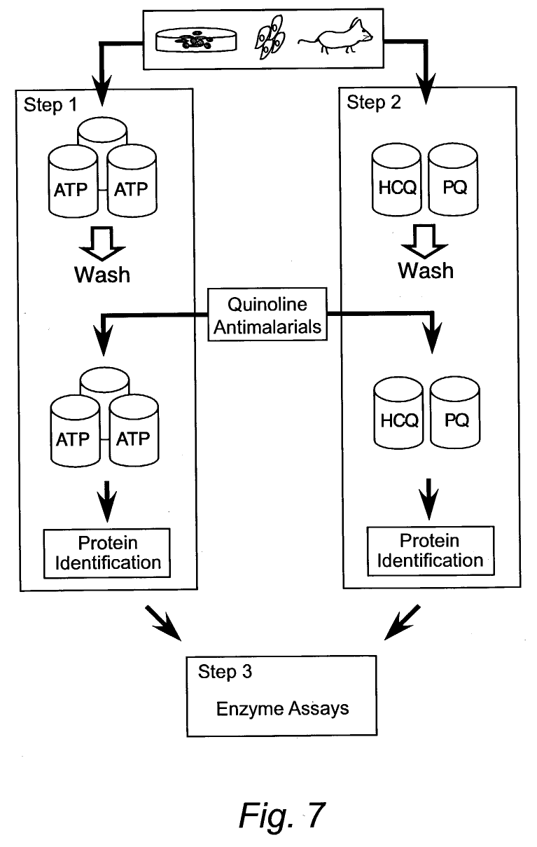

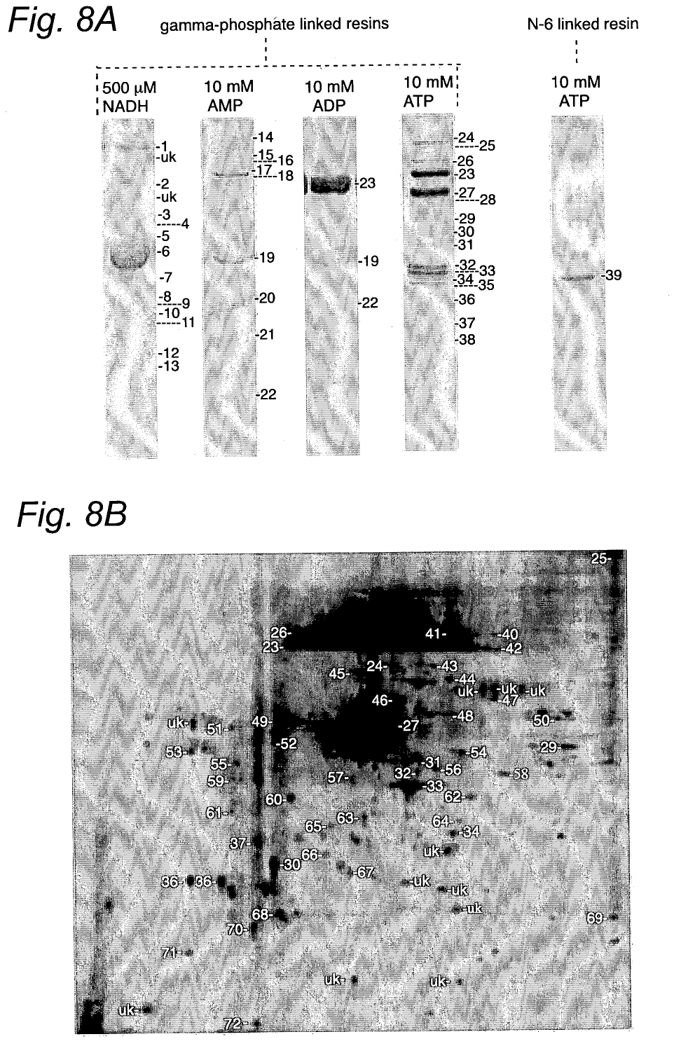

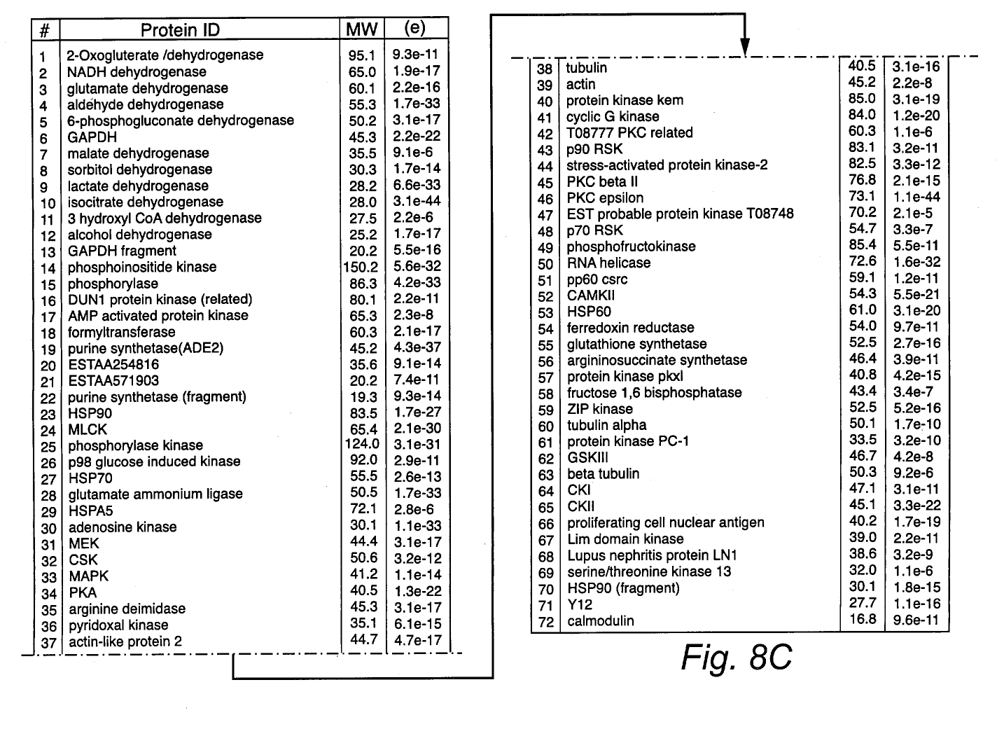

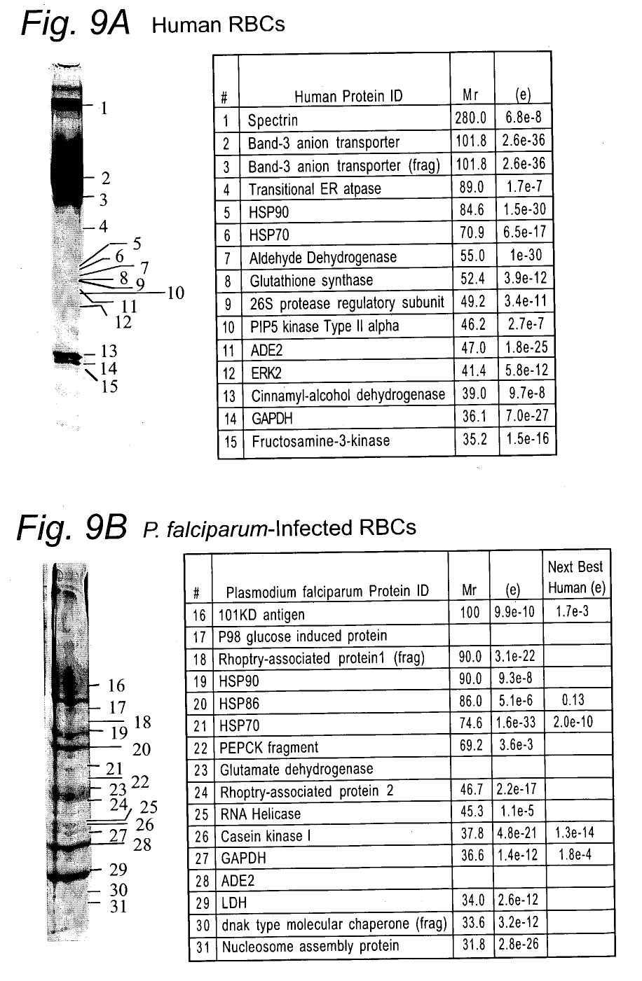

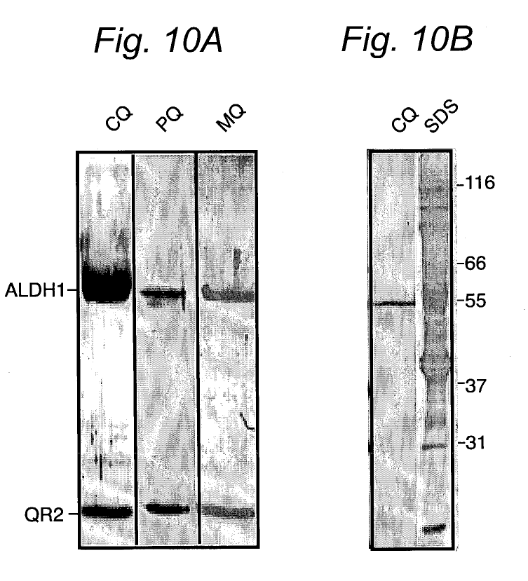

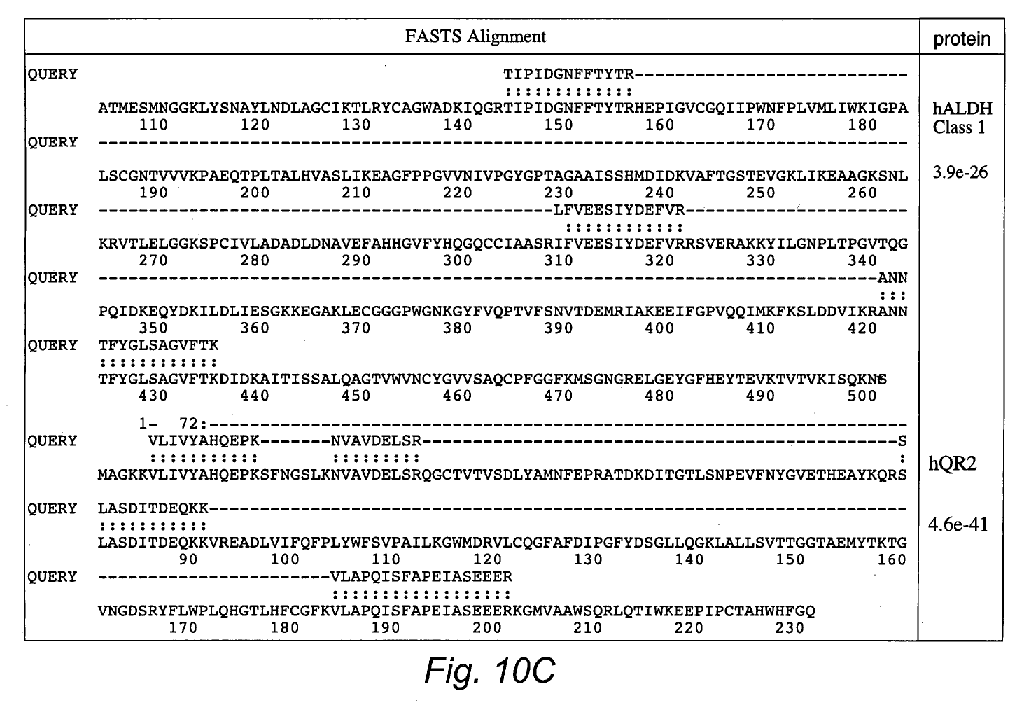

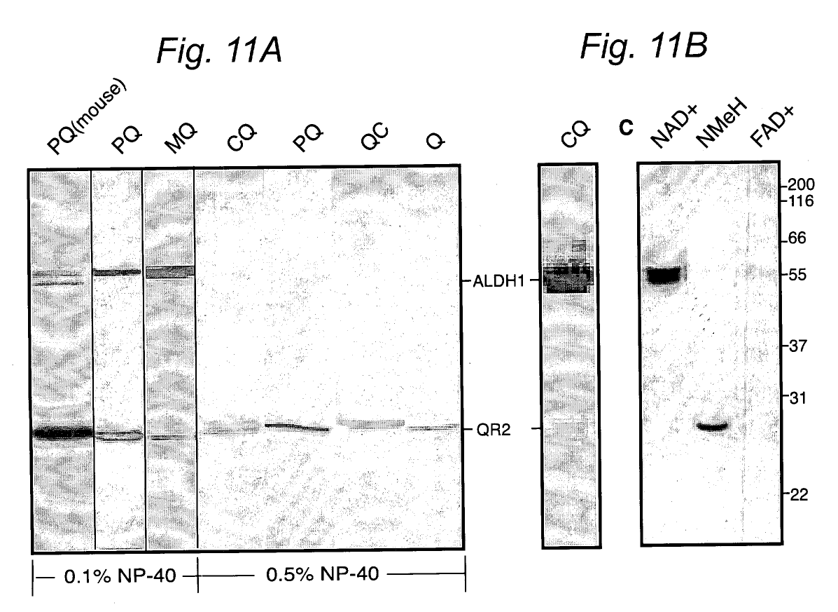

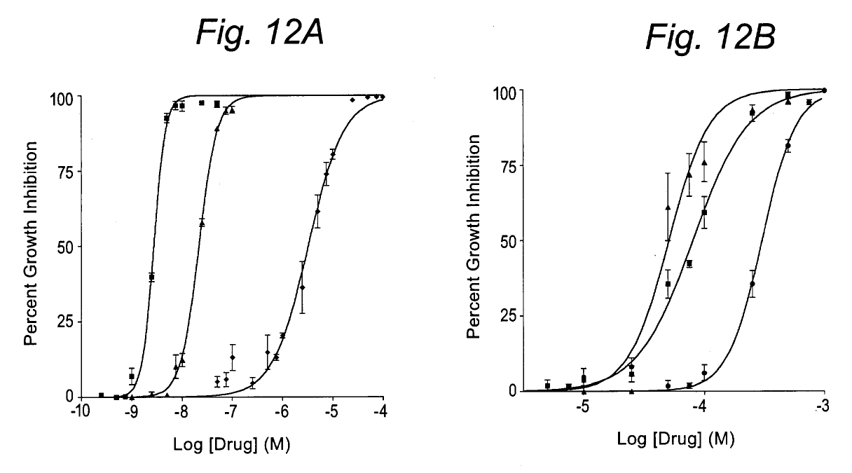

To better understand the mechanism of the quinolines, an attempt was made to identify all quinoline interacting proteins in a cell or animal lysate. To achieve this, a functional proteomics approach was used as outlined in FIG. 7. In this strategy, three different, yet complementary approaches were conducted to identify and validate targets of the quinolines. In step one, termed displacement affinity chromatography, a specific sub-proteome from a cell is captured on an affinity matrix by virtue of its interaction with an immobilized ligand (FIG. 7, Step 1). The sup-proteome is captured after application of saturating amounts of cell lysate and extensive washing of the resin. The compounds of interest (in this case, the quinolines) are then applied to the matrix in parallel and allowed to interact with the bound proteome. If a compound is capable of interacting with a bound protein and can displace it from the affinity matrix, the protein is recovered in the eluent and identified by mass spectrometry. Since the drug presumably has the potential to interact with all of the proteins bound to the matrix, information about drug specificity can be obtained by identification of the eluted proteins (FIG. 7, Step 1). In the second step, affinity matrices were created by directly linking the quinoline drugs to Sepharose, and after application of cell lysates, all proteins that specifically eluted from these matrices in the presence of drug were identified (FIG. 7 Step 2). Finally, in the third step, protein targets identified in the first two steps were assayed for activity in the presence of the quinolines (FIG. 7 Step 3).

In this study, ATP linked to Sepharose via its gamma phosphate group was used to capture the purine binding proteome of cells for subsequent screening with the quinoline drugs (Haystead, Eur. J. Biochem. 2142):459-467 (1993)). To determine the specificity of γ-ATP-Sepharose, the affinity matrix was saturated with extract from a whole homogenized mouse. Following extensive washing to remove non-specific proteins, the resin was sequentially eluted with NADH, AMP, ADP and ATP and the eluted proteins characterized by 1-D or 2-D SDS-PAGE (FIGS. 8A, 8B). Importantly, if ATP was linked to Sepharose through adenosine at N6 (N-6 linked resin) very few proteins were recovered from mouse extract (FIG. 8A).

On average, ˜400 distinct proteins (n=8) were detected in the gels of which 72 were identified by mixed peptide sequencing (Damer et al, J. Biol. Chem. 273(38):24396-24405 (1998)) and mass spectrometry. Examination of the proteins that bound specifically to ATP-Sepharose (FIG. 8C) indicates that a diverse array of purine nucleotide utilizing proteins was recovered. Moreover, the selectivity of ATP-Sepharose for purine binding proteins is demonstrated by the fact that all the proteins sequenced utilize purines or molecules closely resembling purines. Bound proteins identified include protein and non-protein kinases, dehydrogenases, DNA ligases, mononucleotide ATPases, and non-conventional purine binding proteins (FIG. 8C).

To capture the purine binding proteome from human RBCs or P. falciparum, cell extracts from RBCs and P. falciparum parasites were passed in parallel over ATP-Sepharose. Sufficient cell mass (10⁷-10⁸ cells) was applied to each column to saturate all available ATP binding sites and to ensure detection and recovery of proteins expressed at 100 copies/cell (1 fmol). A selection of the bound proteins from each column was sequenced following elution with SDS (FIGS. 9A and 9B). Proteins were identified by FASTF or FASTS (Mackey et al, Molecular and Cellular Proteomics 1.2:139-147 (2002)) database searching algorithms with peptide sequences derived from mixed peptide sequencing or mass spectrometry, respectively. This search strategy was important because RBCs infected with P. falciparum contained a mixture of human and P. falciparum proteins. Using multiple peptide alignments, expectation (e) scores for top scoring P. falciparum proteins ranged from 10⁻⁶ to 10⁻³³ compared to their respective human homologs that generally ranged from 10⁻² to 10⁻⁴ (FIG. 9B). Because of the large diversity of proteins from human RBC and P. falciparum captured on ATP-Sepharose, this matrix is ideal for screening targets of the quinolines.

Identification of quinoline antimalarial binding proteins in the human red blood cell purine binding proteome by displacement affinity interaction. To identify quinoline binding proteins from human RBCS, ATP-Sepharose columns were charged with RBC extracts, washed, and eluted in parallel with 5 mM chloroquine (CQ), primaquine (PQ), and mefloquine (MQ). All three drugs selectively eluted proteins of 55 and 26 kDa (FIG. 10A). The 55 and 26 kDa proteins were sequenced by mass spectrometry and identified as human aldehyde dehydrogenase 1 (ALDH1) [EC 1.2.1.3] and human quinone reductase 2 (QR2) [EC 1.6.99.2] respectively (FIG. 10C). Considering the number of other purine binding proteins captured by ATP-Sepharose from RBCs (FIG. 8A), these data indicate that the quinoline moieties of CQ, PQ, and MQ are highly selective towards ALDH1 and QR2.

To identify quinoline binding proteins from P. falciparum, parasites were isolated from P. falciparum-infected RBCs by saponin lysis (Schlichtherle et al (eds) Methods in Malaria Research 77(MR4/ATCC, Manasas, Va. 2000) and washed extensively to remove RBC proteins. The parasites were lysed, applied to ATP-Sepharose, washed, and eluted with 5 mM CQ. A single protein was detected in the eluate and was identified by mass spectrometry sequencing as human ALDH1 (FIG. 10B). The presence of human ALDH1 in the P. falciparum enriched sample is most likely due to the inability to remove all human RBC proteins from the isolated parasites. No P. falciparum proteins eluted from ATP-Sepharose with 5 mM CQ even though P. falciparum proteins bound ATP-Sepharose (FIGS. 8B and 10B). A search of the annotated P. falciparum genome with multiple peptide sequences derived from human ALDH1 or QR2 did not identify a P. falciparum homolog of these proteins. These findings indicate the presence of two novel targets for the quinoline drugs encoded by the human genome.

Primaquine and chloroquine-Sepharose selectively bind ALDH1 and QR2 from human RBCS. To investigate the selectivity of the quinolines further, PQ and hydroxychloroquine (HCQ) affinity columns were generated. PQ and HCQ were immobilized to Sepharose via their primary amine and hydroxyl group respectively (FIG. 4). This orientation of the immobilized PQ and CQ puts the quinoline moiety in a solvent accessible position. PQ- and HCQ-Sepharose were charged with RBC extracts and eluted with 5 mM PQ or CQ, respectively (FIGS. 11A, 11B). Two major proteins eluted from PQ and HCQ-Sepharose and were identified by microsequencing as human ALDH1 and QR2 (FIGS. 11A, 11B). To explore the specificity of PQ-Sepharose against a more complicated mixture of proteins, whole mouse extract was applied to PQ-Sepharose, washed and then eluted with 5 mM PQ. Three proteins eluted with PQ, and were identified by mass spectrometry as ALDH1, ALDH2, and QR2 (FIG. 11A). To test the strength of interaction between ALDH1, QR2 and PQ-Sepharose, the amount of NP-40 in the wash buffer was increased to 0.5%. Under these more stringent wash conditions, human QR2 was the only protein recovered from human RBCs following elution with CQ, PQ, QC, and Quinine (Q) (FIG. 11A). This result suggests that ALDH1 binds PQ-Sepharose with a lower affinity than QR2. Significantly, when PQ- or HCQ-Sepharose was charged with P. falciparum lysate and eluted with PQ or CQ respectively, no proteins were detected in the eluates.

Inspection of the ALDH1 crystal structure suggests that the NAD⁺ binding pocket is most likely responsible for the interaction of ALDH1 with ATP-Sepharose. This is supported by the elution of ALDH1 from ATP-Sepharose with NADH (FIG. 8A). The NAD⁺ binding pocket is also probably the site where PQ binds ALDH1 since ALDH1 is selectively eluted from PQ-Sepharose with NAD⁺ (FIG. 11C). For QR2, either the adenosine binding pocket of the FAD⁺ moiety or the substrate binding pocket could explain its affinity for PQ-Sepharose. To determine which binding pocket was involved, PQ-Sepharose was charged with RBC extract and eluted with FAD⁺ or the QR2 substrate analog, n-methyldihydronicotinamide (NMeH) (Ortiz-Maldonado et al, Biochemistry 38(50):16636-16647 (1999)) (FIG. 11C). No proteins were eluted with FAD⁺, whereas NMeH eluted QR2, suggesting that the substrate binding pocket of QR2 is the site of interaction with the quinolines.

The quinolines inhibit ALDH1 and QR2. To determine the effect of the quinolines on the activity of ALDH1, ALDH1 was assayed in vitro in the presence of CQ. Because of the co-absorbance of NADH and CQ at 340 nm, an HPLC based assay was developed to determine the effects of CQ on ALDH1 activity. At physiological concentrations of NAD⁺, CQ was a relatively weak inhibitor of ALDH1, with a an IC₅₀ value in the high micromolar range (IC₅₀=500 μM).

To test the ability of the quinolines to inhibit QR2 in vitro, QR2 activity was assayed in the presence of various concentrations of CQ, PQ, QC, MQ and Q. As listed in Table 3, CQ, PQ, and QC were potent inhibitors of QR2 activity. In contrast, MQ and Q, both of which have large bulky substituents at the C-4 position (FIG. 4), are less potent inhibitors of the enzyme (Table 3). The effect of the quinolines on the activity of quinone reductase 1 (QR1), an enzyme that shares 49% amino acid identity with QR2, was also tested. Interestingly, QR1 activity is not affected by CQ and QC and is weakly inhibited by MQ and PQ. These results indicate that the quinolines have specificity within the quinone reductase family of enzymes.

TABLE 3
Effect of antimalarial compounds on the
activity of QR2 and QR1.

| | QR2 | QR1 |
|---|---|---|
| Drug | K1 (μM) ± | IC₅₀ (μM) ± |
| | SEM | SEM |
| Chloroquine | 0.61 ± 0.10 | >1000 |
| Primaquine | 1.04 ± 0.38 | 124 ± 10 |
| Quinacrine | 0.51 ± 0.11 | >1000 |
| Mefloquine | 17.0 ± 4.0 | 616 ± 60 |
| Quinine | 252 ± 50 | 9.6 ± 0.80 |
| Dicumarol | * | 0.175 ± 0.01 |

* QR2 is insensitive to dicumarol (Zhao et al, Proc. Natl. Acad. Sci. USA 95(5): 1669-1674 (1997)).

Effect of QR2 and ALDH1 Inhibitors on P. falciparum Growth

To determine the contribution of QR2 or ALDH1 inhibition to the antimalarial properties of the quinolines, known inhibitors of QR2 or ALDH1 were added to P. falciparum and its growth was measured. Known inhibitors of P. falciparum growth all had IC₅₀ values in agreement with the literature (FIG. 12A). Two specific inhibitors of QR2, quercetin and chrysin, were lethal to the parasites at micromolar concentrations, with IC₅₀ values of 81.8 μM±2.2 and 53.8 μM±6.3, respectively (FIG. 12B). The growth of P. falciparum was also inhibited in vitro by a specific inhibitor of ALDH1, diethylaminobenzaldehyde (DEAB), (FIG. 12B), with an IC₅₀=277 μM±15. Although lethal to P. falciparum, the QR2 and ALDH1 inhibitors did not kill the parasites as effectively as the quinoline compounds. The explanation for this finding is likely to be related to the abilities of the drugs to penetrate the plasma membrane or their ability to become concentrated within P. falciparum infected RBCs.

All documents cited above are hereby incorporated in their entirety by reference.

What is claimed is:

1. A method of screening a test compound for its potential as a therapeutic in a medical indication amenable to treatment with a chloroquine-based drug comprising:
i) contacting said test compound and quinone reductase 2 (QR2), or portion thereof, or fusion protein comprising said QR2 or said portion thereof,
ii) determining the amount of said test compound bound to said QR2, or said portion thereof or said fusion protein,
wherein a test compound that binds QR2, or said portion thereof or said fusion protein, is potentially useful for the treatment of said medical indication.

2. The method according to claim 1 wherein said test compound or said QR2, or said portion thereof or said fusion protein, bears a detectable label.

3. The method according to claim 1 wherein said QR2, or said portion thereof or said fusion protein, is attached to a solid support.

4. The method according to claim 1 wherein said portion comprises a nucleotide binding domain of said QR2.

5. The method according to claim 1 wherein said portion comprises the QR2 active site or cofactor binding site.

6. A method of screening a test compound for its potential as a therapeutic in a medical indication amenable to treatment with a chloroquine-based drug comprising:
i) contacting said test compound and aldehyde dehydrogenase (ALDH), or portion thereof or fusion protein comprising said ALDH or said portion thereof,
ii) determining the amount of said test compound bound to said ALDH, or said portion thereof or said fusion protein,
wherein a test compound that binds ALDH, or said portion thereof or said fusion protein, is potentially useful for the treatment of said medical indication.

7. The method of claim 6 wherein said test compound or said ALDH, or said portion thereof or said fusion protein, bears a detectable label.

8. The method of claim 6 wherein said ALDH, or said portion thereof or said fusion protein, is attached to a solid support.

9. The method according to claim 6 wherein said portion comprises a nucleotide binding domain of said ALDH.

10. The method according to claim 6 wherein said portion comprises the ALDH active site or cofactor binding site.

11. A method of screening a test compound for its potential as a therapeutic in a medical indication amenable to treatment with a chloroquine-based drug comprising:
i) contacting QR2, or portion thereof or fusion protein comprising said QR2 or said portion thereof, with an agent known to bind thereto, under conditions such that said agent can bind to said QR2, or said portion thereof or said fusion protein, wherein said contacting is effected in the presence and absence of said test compound, and
ii) determining the amount of said agent bound to said QR2, or said portion thereof or said fusion protein, in the presence and absence of said test compound,
wherein a reduction in the amount of said agent bound to said QR2, or said portion thereof or said fusion protein, in the presence of said test compound indicates that said test compound has potential as a therapeutic in said medical indication.

12. The method according to claim 11 wherein said agent is chloroquine, mefloquine or primaquine.

13. The method according to claim 11 wherein said agent bears a detectable label.

14. The method according to claim 11 wherein said QR2, or said portion thereof or said fusion protein, is bound to a solid support.

15. A method of screening a test compound for its potential as a therapeutic in a medical indication amenable to treatment with a chloroquine-based drug comprising:
i) contacting ALDH, or portion thereof or fusion protein comprising said ALDH or said portion thereof, with an agent known to bind thereto, under conditions such that said agent can bind to said ALDH, or said portion thereof or said fusion protein, wherein said contacting is effected in the presence and absence of said test compound, and
ii) determining the amount of said agent bound to said ALDH, or said portion thereof or said fusion protein, in the presence and absence of said test compound,
wherein a reduction in the amount of said agent bound to said ALDH, or said portion thereof or said fusion protein, in the presence of said test compound indicates that said test compound has potential as a therapeutic in said medical indication.

16. The method according to claim 15 wherein said agent is chloroquine, mefloquine or primaquine.

17. The method according to claim 15 wherein said agent bears a detectable label.

18. The method according to claim 15 wherein said ALDH, or said portion thereof or said fusion protein, is bound to a solid support.

19. A method of culturing stem cells comprising incubating said cells in a medium comprising a compound identifiable by the method according to claim 6 or 15.

20. A method of treating a medical indication responsive to a chloroquine-based drug, comprising administering to a patient in need of such treatment an amount of a compound identifiable by the method of one of claims 1, 6, 11 and 15, sufficient to effect said treatment, wherein said compound is not chloroquine, quinacrine, quinine, primaquine or mefloquine, or a compound shown in FIG. 4.

21. The method according to claim 20 wherein said indication is malaria, an autoimmune disease or HIV.

22. The method according to claim 21 wherein said indication is rheumatoid arthritis or lupus.
